(19) United States
(12) Patent Application Publication
(10) Pub. No.: US 2022/0346931 A1
(43) Pub. Date: Nov. 3, 2022

(54) DELIVERY SYSTEM FOR A MEDICAL DEVICE

(71) Applicant: ADIENT MEDICAL, INC., Pearland, TX (US)

(72) Inventor: Mitchell Donn EGGERS, Pearland, TX (US)

(21) Appl. No.: 17/731,630

(22) Filed: Apr. 28, 2022

Related U.S. Application Data
(60) Provisional application No. 63/183,166, filed on May 3, 2021.

(51) Int. Cl. A61F 2/01

(52) U.S. Cl. CPC A61F 2/011; A61F 2/0105; A61F 2/0108

(57) ABSTRACT

The present invention relates generally to a method and apparatus for retaining a mechanical connection between an intravascular medical device and a delivery system during deployment until deliberately released in a vessel. In some embodiments, the present invention relates to a method and apparatus for the deployment and retrieval of a vena cava filter.

### FIELD

The present invention relates generally to a method and apparatus for delivering an object in a cavity.

### BACKGROUND

Most conventional intravascular medical devices are deployed without a mechanism that maintains device connection following release from a catheter. Many of these intravascular devices are self-expandable and upon release from a catheter they engage the vessel wall allowing no opportunity for subsequent manipulation by the operator. Situations may occur that require manipulation post unsheathing such as when a vena cava filter is tilted or improperly positioned.

Moreover, if the intravascular medical device is not self-expanding, then often an intravascular balloon is used to ensure vessel apposition. Here a mechanical connection from the device to the delivery system is maintained, during the period of release from a catheter to balloon-assisted vessel apposition, to prevent unwanted device migration during deployment.

In both described situations of self-expandable and non-self-expandable intravascular medical device deployments, it would be desirable for the device to remain connected to the delivery system until the operator is satisfied with device position, then subsequently released.

### SUMMARY

The present invention relates generally to a method and apparatus for retaining a mechanical connection between an intravascular medical device and a delivery system during deployment until deliberately released in a vessel. In some embodiments, the present invention relates to a method and apparatus for the deployment and retrieval of a vena cava filter. An example of such a filter is described in U.S. patent application No. 13/403,790 entitled “Absorbable Vascular Filter” filed Feb. 23, 2012.

Most conventional inferior vena cava (IVC) filters are self-expandable. That is, when released from a catheter, they spring outward and are secured with metallic barbs at the release site in the IVC, with little to no opportunity for repositioning. In contrast, the present invention enables the user to maintain grip of an IVC filter, enabling repositioning of the filter in the IVC following unsheathing of the filter. The present invention also provides an advantage for non-self-expandable IVC filers such as absorbable filters that require balloon-assisted deployment. Here the mechanical connecting maintains grip of the filter during balloon inflation prior to caval apposition.

Thus, according to an embodiment, there is provided a system configured to deliver an intravascular medical device to a location in a vessel. The system comprises a device cartridge that contains the device within a tube that can be routed over a retention wire similar in size to an intravascular guide wire. The system comprises a guiding catheter configured to provide a conduit to and from the location for the device. The system comprises device deployment components configured to facilitate deployment of the device through the guiding catheter at the location in the vessel. The system comprises a retention mechanism formed at a distal end of the retention wire configured to secure the device while the device is in the vessel. The retention mechanism comprises: (i) an outer tube with distal fingers that protrude distally through an opening in a distal end of the device; and (ii) an inner rod or inner tube within the outer tube that prevents the distal fingers on the outer tube from collapsing. Responsive to the inner rod or inner tube being withdrawn proximally with respect to the outer tube, the distal fingers of the outer tube collapse to facilitate withdrawal of the distal fingers through the opening in the distal end of the device, and withdrawal of the inner rod or inner tube and the outer tube from the device, thereby releasing the device at the location within the vessel.

In some embodiments, the device deployment components comprise a balloon catheter than can be routed over the retention wire to expand the device at the location.

In some embodiments, the system further comprises a pusher tube configured to push the device from the cartridge into the guiding catheter.

In some embodiments, the vessel is the vena cava and the device is a vena cava filter.

In some embodiments, the vena cava filter is absorbable. In some embodiments, the vena cava filter is non-self-expandable, but still contained within the device cartridge.

In some embodiments, the device cartridge containing the device is assembled prior to deployment by compressing the device over a support tube, and inserting the device and the support tube into a sheath. In some embodiments, the device cartridge containing the device is configured to be passed over the retention wire through a hemostatic valve of the guiding catheter toward a distal end of the retention wire and the retention mechanism. In some embodiments, the support tube is configured to be removed from a center of the device cartridge such that a pusher tube can be routed over the retention wire to abut the device within the device cartridge, and while the device cartridge is held stationary, the device can be pushed by the pusher tube distally into the guiding catheter. In some embodiments, the pusher tube is configured to be held stationary while the guiding catheter is pulled proximally to unsheath the device such that the device is exposed to the vessel but retained in position with the retention mechanism at the distal end of the retention wire.

According to another embodiment, a method for delivering an intravascular medical device to a location in a vessel with a delivery system is provided. The system comprises a guiding catheter, device deployment components, and a retention mechanism. The method comprises: inserting a retention wire through a guiding catheter; advancing the device over the retention wire into the guiding catheter; unsheathing the device exposing it in the vessel by pulling the guiding catheter proximally; positioning the device in a desired position within the vessel; releasing the device from the retention wire by withdrawing proximally the inner lock rod with respect to the outer retention tube allowing distal fingers of the outer retention tube to collapse to facilitate withdrawal of the distal fingers through the opening in the end of the device; and withdrawing the retention wire from the guiding catheter and finally removing the guiding catheter from the vessel.

### BRIEF DESCRIPTION OF THE DRAWINGS

The above aspects and other aspects and features will become apparent to those ordinarily skilled in the art upon review of the following description of specific embodiments in conjunction with the accompanying figures.

FIG. 1 is a cut-away isometric view of the first of a series of figures (FIGS. 1-7) detailing a method of loading and deploying a non-self-expanding IVC filter using a catheter-based delivery system that maintains connection to the filter throughout deployment. Here the IVC filter is compressed over a support tube and then inserted together with the support tube into a short sheath (to produce a filter cartridge) to facilitate introduction into the guiding catheter.

FIG. 2 is a cut-away isometric view of the second of a series of figures (FIGS. 1-7) detailing a method of loading and deploying a non-self-expanding IVC filter using a catheter-based delivery system that maintains connection to the filter throughout deployment. Here the filter 110 cartridge is inserted into the guiding catheter over a guide wire with retention tip (distal end).

FIG. 3 is a cut-away isometric view of the third of a series of figures (FIGS. 1-7) detailing a method of loading and deploying a non-self-expanding IVC filter using a catheter-based delivery system that maintains connection to the filter throughout deployment. Here the emphasis is on the distal end of the retention wire that maintains connection of the filter to the delivery system.

FIG. 4 is a cut-away isometric view of the fourth of a series of figures (FIGS. 1-7) detailing a method of loading and deploying a non-self-expanding IVC filter using a catheter-based delivery system that maintains connection to the filter throughout deployment. Here a pusher tube is shown allowing advancement of the filter to the distal end of the retention wire.

FIG. 5 is a cut-away isometric view of the fifth of a series of figures (FIGS. 1-7) detailing a method of loading and deploying a non-self-expanding IVC filter using a catheter-based delivery system that maintains connection to the filter throughout deployment. Here the filter is shown unsheathed and residing against the retention wire mechanism at the distal end.

FIG. 6 is a cut-away isometric view of the sixth of a series of figures (FIGS. 1-7) detailing a method of loading and deploying a non-self-expanding IVC filter using a catheter-based delivery system that maintains connection to the filter throughout deployment. Here a balloon catheter is shown expanding the IVC filter while being held by the distal end of the retention wire.

FIG. 7 is a cut-away isometric view of the seventh of a series of figures (FIGS. 1-7) detailing a method of loading and deploying a non-self-expanding IVC filter using a catheter-based delivery system that maintains connection to the filter throughout deployment. Here the filter has been deployed and released from the retention wire.

FIG. 8 is a first magnified view of the distal end of the filter and retention wire showing the step-by-step process of releasing the filter from the retention mechanism in several sequential positions.

FIG. 9 is a second magnified view of the distal end of the filter and retention wire showing the step-by-step process of releasing the filter from the retention mechanism in several sequential positions.

FIG. 10 is a third magnified view of the distal end of the filter and retention wire showing the step-by-step process of releasing the filter from the retention mechanism in several sequential positions.

FIG. 11 is a first magnified view of the distal end of the retention wire during filter release.

FIG. 12 is a second magnified view of the distal end of the retention wire during filter release.

FIG. 13 is a magnified view of the distal end of an alternative retention wire resembling a basket during filter release.

FIG. 14 is a magnified view of the distal end of an alternative retention wire resembling a basket during filter release.

### DETAILED DESCRIPTION

Embodiments of the present invention will now be described in detail with reference to the drawings, which are provided as illustrative examples so as to enable those skilled in the art to practice the invention. Notably, the figures and example below are not meant to limit the scope of the present invention to a single embodiment, but other embodiments are possible by way of interchange of some or all of the described or illustrated elements. Wherever convenient, the same reference numbers will be used throughout the drawings to refer to same or like parts. Where certain elements of these embodiments can be partially or fully implemented using known components, only those portions of such known components that are necessary for an understanding of the present invention will be described, and detailed descriptions of other portions of such known components will be omitted so as not to obscure the invention. In the present specification, an embodiment showing a singular component should not be considered limiting. Rather, the invention is intended to encompass other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the components referred to herein by way of illustration. The terms “proximal” and “distal” are used with reference to the operator of the extraction device. In particular the distal end will be nearest to the object of extraction, while the proximal end will be nearest to the operator.

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

The present invention relates generally to a method and apparatus for retaining a mechanical connection between an intravascular medical device and a delivery system during deployment until deliberately released in a vessel. In some embodiments, the present invention relates to a method and apparatus for the deployment and retrieval of a non-self-expanding absorbable inferior vena cava (IVC) filter.

Although the present invention can be used to load and deploy a plethora of implantable medical devices in a cavity, deployment of an IVC filter intended to prevent pulmonary embolism (PE) is described herein as one example embodiment to illustrate details of the present method and apparatus. The non-self-expanding nature of the present absorbable IVC filter often requires balloon inflation during deployment, which poses new challenges and opportunities for the accompanying delivery systems such as maintaining hold of the filter prior to and during balloon inflation to prevent migration. Consequently, there is a current demand for the novel delivery system described herein that can both accommodate and exploit the unique features of an absorbable IVC filter and/or other non-self-expanding and self-expanding intravascular medical devices.

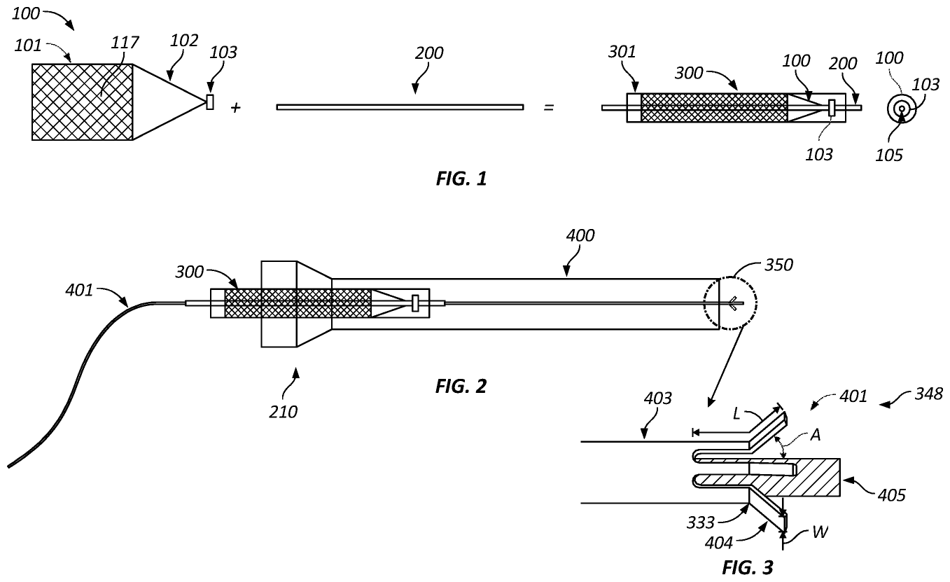

Referring to FIG. 1, an intravascular medical device is shown as an IVC filter 100 comprising a stent section 101 and basket section 102 together with a distal tip or filter end plate 103. The filter end plate 103 has a center hole 105 (an opening) that allows threading the filter 100 over a guide wire or retention wire 401 (shown in FIG. 2). Center hole 105 may be located at a distal end of IVC filter 100. Center hole 105 may be round as shown, or have other shapes. In some embodiments, center hole 105 may be located at or near a central axis of IVC filter 100 and/or in other locations. In some embodiments, center hole 105 may be sized to pass a guidewire and/or retention wire for securing the medical device during deployment.

IVC filter 100 is compressed over a support tube 200 and then together inserted in a short sheath 301 to form a device (filter) cartridge 300. Cartridge 300 is configured to contain IVC filter 100 (e.g., intravascular medical device) within a tube formed by sheath 301 that can be routed over retention wire 401. In other words, filter cartridge 300 containing IVC filter 100 is configured to be assembled prior to deployment by compressing IVC filter 100 over support tube 200, and inserting IVC filter 100 and the support tube 200 into sheath 301.

IVC filter 100 may be an absorbable vascular filter configured to be deployed within a vessel for temporary filtering of body fluids. An embodiment is configured for the placement of such absorbable vascular filter within the IVC for the prevention of pulmonary embolisms for a specific duration of time determined by the absorption properties of the filter.

In some embodiments, the absorbable IVC filter 100 may be configured to slowly biodegrade within the vessel according to a planned schedule engineered by the choice of absorbable filter materials which prevents the requirement of filter removal. In some embodiments, some or all of IVC filter 100 is manufactured from non-metallic synthetic polymers which do not adversely impact end organs upon carefully planned degradation as exhibited by conventional metal IVC filters that migrate and often become fractionated. Also, due to the relative short indwell time (months) of the absorbable vascular filter, the paradoxical increase in DVT seen with conventional long-term IVC filters can be circumvented.

In some embodiments, IVC filter 100 may be laser cut from a tube of absorbable material, for example, and/or formed using other methods. Degradable elements 117 of IVC filter 100 may be purposely designed (e.g., shaped, sized, etc.) to be biologically absorbed and/or degraded after a certain amount of time in a vessel. Degradation can be controlled by the choice of absorbable polymers that possess different absorption profiles, element size, element shape, and/or other factors.

In some embodiments, IVC filter 100 may be formed from a host of biodegradable polymers including polydioxanone, polytrimethylene carbonate, polyglactin, polyglycolic acid, polyglecaprone, polyglytone, and polylacticoglycolic acid.

In some embodiments, IVC filter 100 is non-self-expanding. This means that IVC filter is not formed from a self-expanding metal such as NiTi (Nitinol) and/or self-expanding polymers or other self-expanding materials. IVC filter 100 is not configured to self-expand when exposed to a certain threshold temperature, for example, and is instead configured to be mechanically expanded by a balloon or other device, as described below.

As shown in FIG. 1, IVC filter 100 is configured to be compressed over a support tube 200 and then inserted together with support tube 200 into a short sheath 301 (to produce a filter cartridge 300) to facilitate introduction into a guiding catheter 400 (FIG. 2). Sheath 301 may have a cylindrical tube shape, with a circular cross section. Sheath 301 may be configured to protect IVC filter 100 during delivery and/or have other purposes. For example, the short sheath 301 protects the filter 100 during insertion through a hemostatic or other similar valve (not shown in FIG. 2) located at the proximal end of a guiding catheter 400. In an embodiment applicable to IVC filter deployment, the IVC filter can be compressed to about 4 mm diameter in the filter cartridge that can be inserted into a 12 French (4 mm) guiding catheter for deployment through the femoral vein. Upon balloon expansion the IVC filter would expand to approximately 15 to 30 mm to fit a range of IVC sizes.

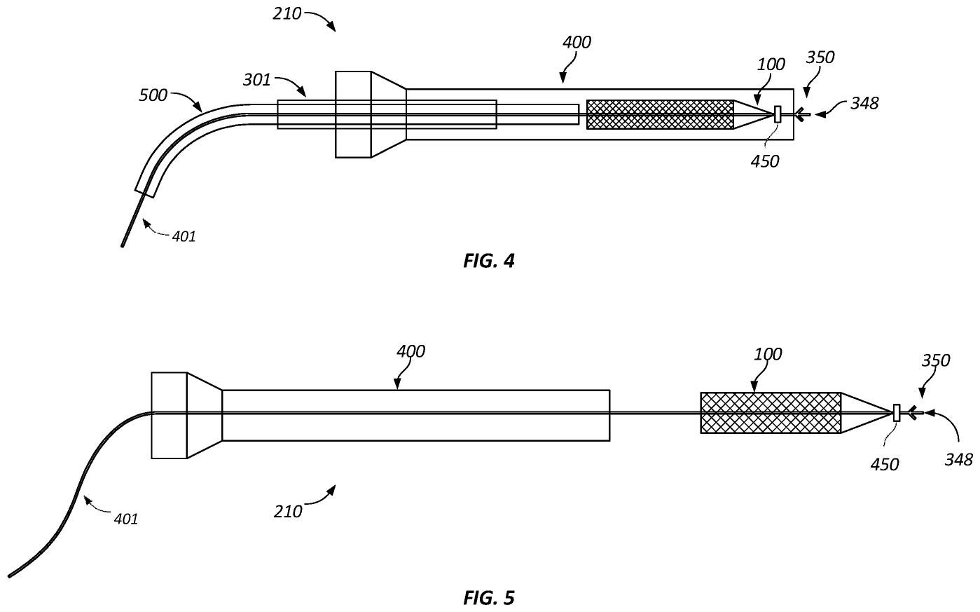

Support tube 200 is configured to be removed from a center of the filter cartridge 300 such that a pusher tube 500 (described below) can be routed over a retention wire 401 (FIG. 4) to abut IVC filter 100 within filter cartridge 300, and while filter cartridge 300 is held stationary, IVC filter 100 can be pushed by the pusher tube distally into guiding catheter 400 (FIG. 4). Support tube 200 may be formed form a polymer, and be relatively light weight so that it is easy to remove, but strong enough to resist being crushed when IVC filter 100 is compressed as described above. The support tube 200 allows the filter 100 to be tightly compressed within the short sheath 301 while maintaining a patent conduit (inside the support tube 200) to readily advance the filter cartridge 300 over the retention wire 401.

Even though IVC filter 100 may not be self-expanding, cartridge 300 may still be configured to contain IVC filter 100. Cartridge 300 may be configured to contain IVC filter 100 while IVC filter 100 is passed through a hemostatic valve into a guide catheter, for example, and/or through other deployment components. Cartridge 300 may be configured to contain IVC filter 100 while IVC filter 100 is advanced toward the distal end of retention wire 401. This may prevent damage to IVC filter 100 and/or have other advantageous effects. For example, an additional advantage of storing the filter in a cartridge is to conserve environmentally-controlled storage space. That is, rather than storing the absorbable filter in its normal expanded state (approximately 15-30 mm dia.) in a refrigerator to prevent premature degradation, the filter can be loaded in the cartridge that only requires 4 mm dia.

FIG. 2 depicts a delivery system 210 and the advancement of the filter cartridge 300 over the retention wire 401 within guiding catheter 400. Delivery system 210 is configured to deliver IVC filter 100 (an intravascular medical device) to a location in a vessel. The vessel may be the vena cava for example. The location may be inferior to the renal veins. Delivery system 210 includes various device deployment components as illustrated in the figures and described herein configured to facilitate deployment of IVC filter 100 through guiding catheter 400 at the location in the vessel.

310 Guiding catheter 400 is configured to provide a conduit to and from the deployment location in the vessel for IVC filter 100. Guiding catheter 400 may be formed as a relatively long, hollow tube. Guiding catheter 400 may be formed from a biocompatible polymer, and/or other materials. Retention wire 401 may be a flexible metal wire that is configured to be tracked through a patient's vasculature. Retention wire 401 may have a relatively small diameter, configured to facilitate the passage of other device deployment components over retention wire 401 to a deployment location. For example, for IVC filter 100 deployment, the delivery system 210 may be inserted into the patient's vasculature of convenient location, such as the femoral vein or internal jugular. Subsequently, the delivery system 210 is fed through the vasculature typically over a wire such as retention wire 401 or other guide wire until reaching the desired deployment location, often inferior to the renal veins.

FIG. 3 illustrates a magnified view of the distal end 350 of the retention wire 401. The distal end 350 of the retention wire 401 forms a retention mechanism 348 configured to secure IVC filter 100 while IVC filter 100 is in a vessel. Retention mechanism 348 comprises an outer retention tube 403 with distal fingers 404 that are configured to protrude distally through center hole 105 (FIG. 1) in a distal end of IVC filter 100. Retention mechanism 348 comprises an inner wire, inner rod, or inner tube (e.g., a lock rod 405) within the outer retention tube 403 that prevents the distal fingers 404 on the outer retention tube 403 from collapsing. For example, as shown in FIG. 3, retention wire 401 comprises an outer retention tube 403 with flared distal fingers 404 and a lock rod 405 residing inside the retention tube 403.

In some embodiments, two, three, four, five, six, or more flared distal fingers 404 are formed at the distal end of retention tube 403. Flared distal fingers 404 may have varying lengths L and/or widths W configured such that flared distal fingers 404 are able to function as described. Flared distal fingers 404 may generally have the same thickness as retention tube 403.

Flared distal fingers 404 flare outward with respect to retention tube 403. Phrased another way, flared distal fingers 404 flare away from a longitudinal centerline of retention tube 403. In some embodiments flaring comprises pivoting, bending, flexing, and/or other movement. In some embodiments, flared distal fingers 404 are configured to pivot, bend, flex, or otherwise move outwardly at a joint 333 formed along the length L of each flared distal finger 404. This joint may be a fold, a scribe line, a divot, a thinned portion, and/or other joints. An angle, A, formed between a finger and the rest of retention tube 403 may be configured to facilitate eventual passage of fingers 404 through end plate 103 (FIG. 1) of IVC filter 100 (FIG. 1). For example, if angle A is too steep, fingers 404 may catch and hold on end plate 103, instead of passing through end plate 103 as described below.

Lock rod 405 is configured to be in sliding engagement with retention tube 403. The sliding engagement facilitates relative movement between lock rod 405 and retention tube 403 when lock wire is moved proximally (or distally) relative to retention tube 403 by an operator (e.g., a doctor).

FIG. 4 illustrates the advancement of a pusher tube 500 configured to push the IVC filter 100 through the short sheath 301 and out of cartridge 300, while IVC filter 100 is still within the guiding catheter 400, until a filter end plate 103 resides against the distal end 350 of the retention wire 401. For example, pusher tube 500 is configured to push IVC filter 100 from the cartridge 300 into the guiding catheter 400 as shown.

Pusher tube 500 may have a tubular shape configured to pass over retention wire 401, and just within sheath 301. Once within sheath 301, pusher tube 500 abuts a proximal end of IVC filter 100. Pusher tube 500 is configured to be held stationary (by an operator) while the guiding catheter 400 is pulled proximally to unsheath IVC filter 100 such that IVC filter 100 is exposed to the vessel but retained in position with the retention mechanism 348 at the distal end 350 of the retention wire 401.

The lock rod 405 within the retention tube 403 prevents the flared retention fingers 404 from collapsing, thereby preventing further distal advancement of the filter end plate 103 over the retention wire 401. Such mechanism thereby maintains IVC filter 100 connection to the delivery system 210.

FIG. 5 illustrates the position of the IVC filter 100 relative to the guiding catheter 400 once the pusher tube 500 is held fixed (pinned) while the guiding catheter 400 is slid proximal to effectively un-sheath the IVC filter 100. Here again the IVC filter 100 is prevented from moving beyond the distal end 350 of the retention wire 401 by the retention mechanism formed by the flared retention fingers 404 through the filter end plate 103 held in place by the lock rod 405.

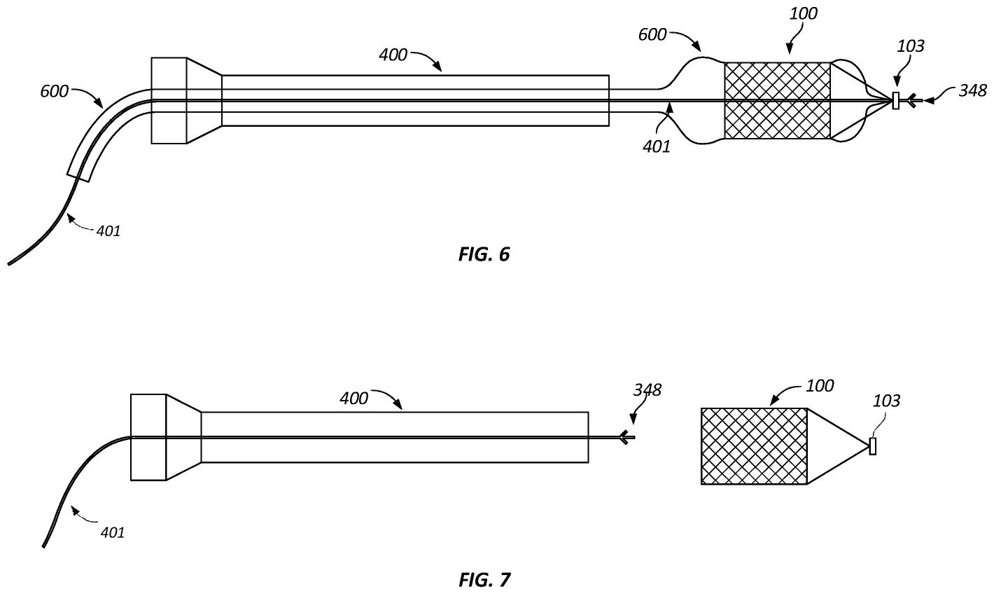

FIG. 6 depicts a balloon catheter 600 (e.g., part of the device deployment components) that has been advanced or otherwise routed over the retention wire 401 and through the hemostatic valve of the guiding catheter 400 to reside within the IVC filter 100. The balloon catheter 600 is shown fully inflated, causing a bulge to occur outside the stent section 101 of the filter.

FIG. 7 depicts deflation and removal of the balloon catheter 600 over the retention wire 401 in addition to severing the connection from the filter 100 to the delivery system. Once the operator has confirmed correct positioning of the filter 100 following balloon-assisted caval apposition, the retention wire 401 is removed per the procedure described below and illustrated in FIGS. 8-10.

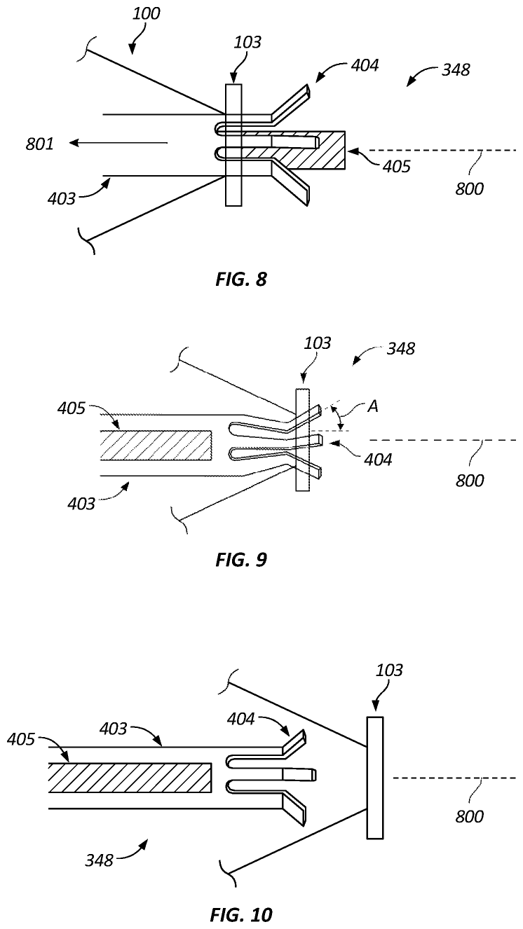

FIGS. 8-10 depict the process of releasing the IVC filter 100 from the retention wire 401. The filter 100 is released from the retention wire by (i) first pulling the proximal end of the lock rod 405 while pinning the retention tube 403 allowing the distal end of the lock rod 405 to retract inside the retention tube 403, thereby allowing the flared retention fingers 404 to collapse (FIG. 9), then (ii) pulling proximal both the lock rod 405 and retention tube 403 simultaneously to enable the entire retention wire 401 to withdraw from the filter end plate 103 (FIG. 10) thereby releasing the filter.

Phrased another way, responsive to the inner rod or inner tube (e.g., lock rod 405) being withdrawn proximally with respect to the outer retention tube 403, the flared distal retention fingers 404 of the outer retention tube 403 collapse to facilitate withdrawal of the distal fingers 404 through the center hole 105 (opening) in the distal end of IVC filter 100, and withdrawal of the inner rod or inner tube (e.g., lock rod 405) and the outer retention tube 403 from IVC filter 100, thereby releasing IVC filter 100 at a location within a vessel.

For example, as shown in FIG. 8, lock rod 405 is beginning to be withdrawn proximally 801 relative to outer retention tube 403 and fingers 404. Because the distal end of lock rod 405 has not yet passed through fingers 404 and/or filter end plate 103, fingers 404 are not yet able to move centrally toward a center axis 800 of IVC filter 100.

FIG. 9 illustrates the distal end of lock rod 405 having passed proximally through fingers 404 and filter end plate 103. In addition, retention tube 403 has also been withdrawn proximally relative to IVC filter 100. Thus, fingers 404 are now able to move centrally toward center axis 800 of IVC filter 100. Movement may comprise flexing, bending, pivoting, and/or other movement, for example. Because of the angle, A, formed between a finger 404 and the rest of retention tube 403, fingers 404 are compressed toward center axis 800 by end plate 103 as fingers pass through end plate 103. If angle A was too steep, fingers 404 may catch and hold on end plate 103, instead of passing through end plate 103.

FIG. 10 illustrates fingers 404 having passed completely through end plate 103 and returned to an original, uncompressed position. Retention tube 403 may be removed from IVC filter 100 with fingers 404 in this position.

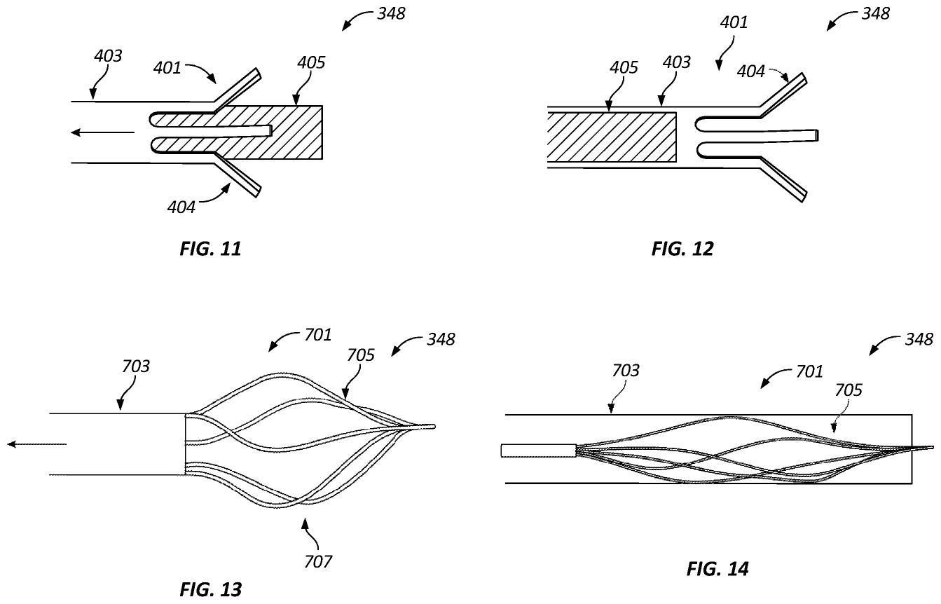

FIGS. 11-12 illustrate a magnified view of the release mechanism of fingers 404 with the lock rod 405 being pulled proximal within the retention tube 403 allowing the retention fingers 404 to bend, compress, or otherwise collapse when pulled through the filter end plate 103. For example, FIG. 11 is similar to FIG. 8, but without IVC filter 100 or end plate 103. FIG. 12 is similar to FIG. 10, but again without IVC filter 100 or end plate 103.

FIGS. 13-14 illustrate a magnified view of an alternative retention wire 701 with a lock rod that is a compressed woven wire 705 that when advanced outward from the retention tube 703 expands to prevent advancement of the filter end plate 103 past the retention wire 701 distal end thereby holding the IVC filter 100 (see FIG. 8-10) in place.

Similar to the mechanics of the fingers 404 described above, the woven wire 705 may form an expanded bulb 707 or other shape that has a larger cross sectional size than filter end plate 103 on a distal side of end plate 103. Woven wire 705 may release filter 100 when retention wire 701 is pulled proximal within the retention tube 703 allowing the bulb of the woven wire 705 to bend, compress, or otherwise collapse when pulled through the filter end plate 103.

An example method of utilizing the described apparatus for retaining a mechanical connection between an intravascular medical device and a delivery system during deployment until deliberately released in a vessel is described below. This example method applies to deploying a non-self-expanding inferior vena cava filter.

Operation 1-Following the Seldinger technique, insert a dilator into the guiding catheter 400 and then advance the dilator and the guiding catheter together over a guide wire as a unit under fluoroscopic guidance to reach the inferior vena cava from the femoral vein.

Operation 2-Under fluoroscopy, continue to advance the guiding catheter 400 with dilator into the IVC until the distal end of the guiding catheter 400 is positioned just inferior to the renal veins.

Operation 3-Remove the dilator and guide wire from the guiding catheter 400 while stabilizing the guiding catheter 400.

Operation 4-Insert the retention wire 401 (which includes retention tube 403 with flared fingers 404 at distal end together with internal lock rod 405) through the guiding catheter 400 hemostatic valve and advance to the distal end.

Operation 5-Advance the filter cartridge 300 containing the IVC filter 100 over the retention wire 401 into the hemostatic valve of the guiding catheter 400 until it is protruding about a finger's width proximal to the guiding catheter 400. The filter cartridge 300 would have been assembled prior to deployment by compressing the filter 100 over a support tube 200 and inserting together in a short sheath 301.

Operation 6-Remove the support tube 200 from the center of the filter cartridge 300 allowing it to slide off the retention wire 401.

Operation 7-Route the pusher tube 500 over the retention wire 401 to abut the filter 100 within the filter cartridge 300. While holding the filter cartridge 300 stationary, push the filter 100 distal into the guiding catheter 400 to the distal end.

Operation 8-While pinning the pusher tube 500 (holding the pusher stationary), pull the guiding catheter 400 proximal to unsheath the filter 100. The filter 100 is now exposed in the IVC and retained in position with the distal end of the retention wire 401. Remove the pusher tube 500.

Operation 9-Advance a balloon catheter 600 over the retention wire 401 and through the guiding catheter 400 hemostatic valve to abut the tip of the filter 100.

Operation 10-Fully inflate the radiopaque contrast-filled balloon of the balloon catheter 600 under fluoroscopy with a syringe. As the balloon of balloon catheter 600 is inflated, filter/caval apposition is achieved when a compliant or semi-compliant balloon forms a “dog bone” shape about the stent section 101 of the filter 100. Close the luer valve on the balloon catheter 600 to maintain pressure.

Operation 11-Extract the retention wire 401 from the filter 100 by (i) first pulling just the lock rod 405 proximal at the proximal end while pinning the retention wire 401, and then (ii) pulling the combined retention wire 401 with lock rod 405 proximal allowing the collapsed retention fingers 404 to detach from the filter 100. Remove retention wire 401 from the guiding catheter 400.

Operation 12-Fully deflate the balloon of the balloon catheter 600 by opening the luer valve and pulling vacuum with a syringe. Slowly pull balloon catheter 600 proximal into the guiding catheter 400 and remove all delivery system components from patient.

Various embodiments of the present systems and methods are disclosed in the subsequent list of numbered clauses. In the following, further features, characteristics, and exemplary technical solutions of the present disclosure will be described in terms of clauses that may be optionally claimed in any combination:

1. A system configured to deliver an intravascular medical device to a location in a vessel, the system comprising: a device cartridge that contains the device within a tube that can be routed over a retention wire; a guiding catheter configured to provide a conduit to and from the location for the device; device deployment components configured to facilitate deployment of the device through the guiding catheter at the location in the vessel; and a retention mechanism formed at a distal end of the retention wire configured to secure the device while the device is in the vessel, the retention mechanism comprising: (i) an outer tube with distal fingers that protrude distally through an opening in a distal end of the device; and (ii) an inner rod or inner tube within the outer tube that prevents the distal fingers on the outer tube from collapsing; wherein, responsive to the inner rod or inner tube being withdrawn proximally with respect to the outer tube, the distal fingers of the outer tube collapse to facilitate withdrawal of the distal fingers through the opening in the distal end of the device, and withdrawal of the inner rod or inner tube and the outer tube from the device, thereby releasing the device at the location within the vessel.

2. The system of clause 1, wherein the device deployment components comprise a balloon catheter than can be routed over the retention wire to expand the device at the location.

3. The system of any of the previous clauses, further comprising a pusher tube configured to push the device from the cartridge into the guiding catheter.

4. The system of any of the previous clauses, wherein the vessel is the vena cava and the device is a vena cava filter.

5. The system of any of the previous clauses, wherein the vena cava filter is absorbable.

6. The system of any of the previous clauses, wherein the vena cava filter is non-self-expandable, but still contained within the device cartridge.

7. The system of any of the previous clauses, wherein the device cartridge containing the device is assembled prior to deployment by compressing the device over a support tube, and inserting the device and the support tube into a sheath.

8. The system of any of the previous clauses, wherein the device cartridge containing the device is configured to be passed over the retention wire through a hemostatic valve of the guiding catheter toward a distal end of the retention wire and the retention mechanism.

9. The system of any of the previous clauses, wherein the support tube is configured to be removed from a center of the device cartridge such that a pusher tube can be routed over the retention wire to abut the device within the device cartridge, and while the device cartridge is held stationary, the device can be pushed by the pusher tube distally into the guiding catheter.

10. The system of any of the previous clauses, wherein the pusher tube is configured to be held stationary while the guiding catheter is pulled proximally to unsheath the device such that the device is exposed to the vessel but retained in position with the retention mechanism at the distal end of the retention wire.

11. A method for delivering an intravascular medical device to a location in a vessel with a delivery system, the system comprising a guiding catheter, device deployment components, and a retention mechanism, the method comprising: inserting a retention wire through a guiding catheter; advancing the device over the retention wire into the guiding catheter; unsheathing the device exposing it in the vessel by pulling the guiding catheter proximally; positioning the device in a desired position within the vessel; releasing the device from the retention wire by withdrawing proximally the inner lock rod with respect to the outer retention tube allowing distal fingers of the outer retention tube to collapse to facilitate withdrawal of the distal fingers through the opening in the end of the device; and withdrawing the retention wire from the guiding catheter and finally removing the guiding catheter from the vessel.

12. The method of any of the previous clauses, wherein the device deployment components comprise a balloon catheter configured to expand the device at the location, and the method further comprises deploying the device with the balloon.

13. The method of any of the previous clauses, wherein a pusher tube is used to push the device into the guiding catheter.

14. The method of any of the previous clauses, wherein the intravascular device is an inferior vena cava filter and the vessel is the inferior vena cava.

15. The method of any of the previous clauses, wherein the device is pre-loaded within a sheath to form a device cartridge that prevents damage to the device upon insertion into the guiding catheter.

16. The method of any of the previous clauses, wherein the device cartridge containing the device is assembled prior to deployment by compressing the device over a support tube, and inserting the device and the support tube into the sheath.

17. The method of any of the previous clauses, wherein the device cartridge containing the device is configured to be passed over the retention wire through a hemostatic valve of the guiding catheter toward a distal end of the retention wire and the retention mechanism.

18. The method of any of the previous clauses, wherein the support tube is configured to be removed from a center of the device cartridge such that a pusher tube can be routed over the retention wire to abut the device within the device cartridge, and while the device cartridge is held stationary, the device can be pushed by the pusher tube distally into the guiding catheter.

19. The method of any of the previous clauses, wherein the pusher tube is configured to be held stationary while the guiding catheter is pulled proximally to unsheath the device such that the device is exposed to the vessel but retained in position with the retention mechanism at the distal end of the retention wire.

20. The method of any of the previous clauses, wherein the device is a vena cava filter, wherein the vena cava filter is absorbable, and wherein the vena cava filter is non-self-expandable, but still contained within a device cartridge.

21. The method of any of the previous clauses, performed at least in part using the system of any of the previous clauses.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

What is claimed is:

1. A system configured to deliver an intravascular medical device to a location in a vessel, the system comprising:
a device cartridge that contains the device within a tube that can be routed over a retention wire;
a guiding catheter configured to provide a conduit to and from the location for the device;
device deployment components configured to facilitate deployment of the device through the guiding catheter at the location in the vessel; and
a retention mechanism formed at a distal end of the retention wire configured to secure the device while the device is in the vessel, the retention mechanism comprising:
(i) an outer tube with distal fingers that protrude distally through an opening in a distal end of the device; and
(ii) an inner rod or inner tube within the outer tube that prevents the distal fingers on the outer tube from collapsing;
wherein, responsive to the inner rod or inner tube being withdrawn proximally with respect to the outer tube, the distal fingers of the outer tube collapse to facilitate withdrawal of the distal fingers through the opening in the distal end of the device, and withdrawal of the inner rod or inner tube and the outer tube from the device, thereby releasing the device at the location within the vessel.

2. The system of claim 1, wherein the device deployment components comprise a balloon catheter than can be routed over the retention wire to expand the device at the location.

3. The system of claim 1, further comprising a pusher tube configured to push the device from the cartridge into the guiding catheter.

4. The system of claim 3, wherein the vessel is a vena cava and the device is a vena cava filter.

5. The system of claim 4, wherein the vena cava filter is absorbable.

6. The system of claim 4, wherein the vena cava filter is non-self-expandable, but still contained within the device cartridge.

7. The system of claim 1, wherein the device cartridge containing the device is assembled prior to deployment by compressing the device over a support tube, and inserting the device and the support tube into a sheath.

8. The system of claim 7, wherein the device cartridge containing the device is configured to be passed over the retention wire through a hemostatic valve of the guiding catheter toward a distal end of the retention wire and the retention mechanism.

9. The system of claim 8, wherein the support tube is configured to be removed from a center of the device cartridge such that a pusher tube can be routed over the retention wire to abut the device within the device cartridge, and while the device cartridge is held stationary, the device can be pushed by the pusher tube distally into the guiding catheter.

10. The system of claim 9, wherein the pusher tube is configured to be held stationary while the guiding catheter is pulled proximally to unsheath the device such that the device is exposed to the vessel but retained in position with the retention mechanism at the distal end of the retention wire.

11. A method for delivering an intravascular medical device to a location in a vessel with a delivery system, the system comprising a guiding catheter, device deployment components, and a retention mechanism, the method comprising:
inserting a retention wire through a guiding catheter;
advancing the device over the retention wire into the guiding catheter;
unsheathing the device exposing it in the vessel by pulling the guiding catheter proximally;
positioning the device in a desired position within the vessel;
releasing the device from the retention wire by withdrawing proximally an inner lock rod of the retention mechanism with respect to an outer retention tube of the retention mechanism allowing distal fingers of the outer retention tube to collapse to facilitate withdrawal of the distal fingers through an opening in a distal end of the device; and
withdrawing the retention wire from the guiding catheter and finally removing the guiding catheter from the vessel.

12. The method of claim 11, wherein the device deployment components comprise a balloon catheter configured to expand the device at the location, and the method further comprises deploying the device with the balloon.

13. The method of claim 11, wherein a pusher tube is used to push the device into the guiding catheter.

14. The method of claim 11, wherein the intravascular device is an inferior vena cava filter and the vessel is the inferior vena cava.

15. The method of claim 11, wherein the device is pre-loaded within a sheath to form a device cartridge that prevents damage to the device upon insertion into the guiding catheter.

16. The method of claim 15, wherein the device cartridge containing the device is assembled prior to deployment by compressing the device over a support tube, and inserting the device and the support tube into the sheath.

17. The method of claim 16, wherein the device cartridge containing the device is configured to be passed over the retention wire through a hemostatic valve of the guiding catheter toward a distal end of the retention wire and the retention mechanism.

18. The method of claim 17, wherein the support tube is configured to be removed from a center of the device cartridge such that a pusher tube can be routed over the retention wire to abut the device within the device cartridge, and while the device cartridge is held stationary, the device can be pushed by the pusher tube distally into the guiding catheter.

19. The method of claim 18, wherein the pusher tube is configured to be held stationary while the guiding catheter is pulled proximally to unsheath the device such that the device is exposed to the vessel but retained in position with the retention mechanism at the distal end of the retention wire.

20. The method of claim 11, wherein the device is a vena cava filter, wherein the vena cava filter is absorbable, and wherein the vena cava filter is non-self-expandable, but still contained within a device cartridge.
